(19) United States
(12) Patent Application Publication
(10) Pub. No.: US 2006/0251708 A1
(43) Pub. Date: Nov. 9, 2006

(54) Coenzyme q10 containing proliposome and preparation thereof

(76) Inventors: Jianming Chen, SHANGHAI (CN); Shen Gao, Shanghai (CN); Huiliang Li, Shanghai (CN); Huifen Lin, Shanghai (CN); Shaomin Wei, Shanghai (CN); Yangmei Zhang, Shanghai (CN); Luo Lu, Shanghai (CN); Yangiang Zhong, Shanghai (CN); Qing Shi, Shanghai (CN); Yiguang Guo, Shanghai (CN); Fei Guan, Shanghai (CN); Wei Wang, Shanghai (CN); Laiji Ma, Shanghai (CN); Juan Gu, Shanghai (CN)

Correspondence Address: BUTZEL LONG, 350 SOUTH MAIN STREET, SUITE 300, ANN ARBOR, MI, 48104, US

(21) Appl. No.: 10/549,987

(22) PCT Filed: Mar. 22, 2004
(86) PCT No.: PCT/CN04/00234

§ 371 Date: Sep. 19, 2005

(30) Foreign Application Priority Data
Mar. 20, 2003 (CN) 03115914.1

(51) Int. Cl. A61K 9/127; A61K 38/43

(52) U.S. Cl. 424/450; 424/94.1

(57) ABSTRACT

A coenzyme Q₁₀ and ceramide-containing preliposome, and pharmaceutical preparations and cosmetics containing the same. The preliposome can further comprise other lipid components. A granular and lyophilized solid-preparation is produced by lyophilization or spray drying. Then, the coenzyme Q₁₀-containing proliposome is obtained by adding water in the said solid-preparation and shaking. Transdermal absorption of coenzyme is improved together with the effect of coenzyme Q₁₀ in cosmetics and the stability of coenzyme Q₁₀ and liposome, which facilitate the formulation of cosmetics.

### FIELD OF THE INVENTION

The present invention relates to the fields of pharmaceutics and cosmetics. More specially, the present invention relates to CoQ₁₀-containing preliposomes, and more particularly, relates to the preparation method and the application of CoQ₁₀-containing preliposomes which contain spongiamine.

### BACKGROUND OF THE INVENTION

CoQ₁₀ (conenzymeQ₁₀, ubidecarenone) is a kind of a liposoluble quinine compound, which has the same character as a vitamin. The prominent function of CoQ₁₀ is anti-oxidation and cleaning free radicals CoQ₁₀ is one of the most important functional components used in many anti-aging products at parent. It has been proved experimentally that CoQ₁₀ can accelerate the metabolism of skin, accelerate the transport of cellular respiration chain and ATP production of facial and hand skin. Simultaneously, CoQ₁₀ can inhibit oxidation of the skin lipid, and consequently nourish and activate the skin. It is reported that body slimming lotions and UV expert creams which contain CoQ₁₀ have obvious effects on preventing the formation of furrows, whitening the complexion, increasing the elasticity of the skin and so on. CoQ₁₀ not only protects the skin, but also prevents and cures skin diseases of human beings. It has been experimentally proven that CoQ₁₀ has obvious therapeutic effects on photoallergy, dermatitis, hair-lose, bedsores, ulcers, wounds of the skin, hyperpigmentation and so on. Because the molecular structure of CoQ₁₀ has an unsaturated double bond, CoQ₁₀ is extremely unstable and is easy to oxidize and becomes decomposed by the oxygen and light in the air. In addition, heating or contacting CoQ₁₀ with metal ions will accelerate its decomposition. As a result, the content of CoQ₁₀ in products becomes decreased, and the activity of CoQ₁₀ is quickly lost, adversely affecting the quality and actual effect of the products. In addition, CoQ₁₀ is a liposoluble compound, which makes it difficult to mix with the water-soluble cosmetics. The foregoing disadvantages of CoQ₁₀ extremely restrict the development and application of CoQ₁₀.

Liposomes are composed of hydrophilic bursa bubbles which consist of lecithoid double molecular layers. Liposomes have characteristics that improve the stability of drug encapsulation, facilitate the percutaneons absorption of drugs, prolong the time of drug action, control the drug targeting at local pathological parts of the body, and decrease the side effects of drugs. Thus, as drug carriers, liposomes have been widely used in pharmaceutics and cosmetics. CoQ₁₀ liposomes could improve the stability of drugs, facilitate the percutaneous absorption of drugs, and increase the water-solubility of drugs. But generally being a kind of liposome suspension solution, CoQ₁₀ has obvious shortcomings in the stability. The reasons are as following:

1. As colloidial particulates, liposomes are a kind of unstable thermodynamic system which is easy to congregate, fuse and sedimentate, and the oxidation decompose of the lecithoid causes leakage of the encapsulation drug into the aqueous solution, etc., resulting in the instability of the liposome.

2. The instability of the structure of CoQ₁₀ makes drugs more instable in the water.

3. The ratio of CoQ₁₀, liposome suspension and drug content is generally fixed; however, the required content of CoQ₁₀ differs in different cosmetics. Thus, it is not convenient to mix CoQ₁₀ liposome suspensions with cosmetics which contain CoQ₁₀.

So it is necessary to find a kind of liposome preparation which is convenient, flexible, easy to mix with cosmetics which contain CoQ₁₀, in order to make the liposomes and drugs more stable, and storable for a long periods of time.

### DISCLOSURE OF THE INVENTION

An object of present invention is to overcome the shortcomings of CoQ₁₀ and common CoQ₁₀ liposome, and to supply a kind of CoQ₁₀-containing preliposomes which contain spongiamine. The present invention will increase the stability of CoQ₁₀ and liposomes and make the mixing of cosmetics more flexible and convenient.

The CoQ₁₀-containing preliposomes made according to the present invention are a kind of solid preparation which are the granular and lyophilized. Before using, water is added to the CoQ₁₀-containing preliposomes. After hydration and surging, the CoQ₁₀-containing preliposomes can become CoQ₁₀-containing liposomes.

The structure of the CoQ₁₀-containing preliposomes of the present invention contain spongiamine at a concentration at 0.1%˜20% (W/W). Spongiamine can further facilitate the percutaneous absorption and improve the effect of CoQ₁₀ in cosmetics.

The CoQ₁₀-containing preliposomes which contain spongiamine according to the present invention are prepared by the following methods and processes.

1) CoQ₁₀, spongiamine and other lipid components are melted by heating or are dissolved by proper organic solvent(s) so that a lipid solution is made,

2) A fluidized bed can be used to spray the abovementioned lipid solution on an underlay which is suspended in the middle of the fluidized bed. The organic solvent is volatilized, and CoQ₁₀-containing preliposomes which contain spongiamine is obtained,

3) Make the lipid solution mentioned in step 1) and water solution which contains an underlay by known methods such as a membrane dispersion method or a melt method or an infuse method to obtain CoQ₁₀-containing liposomes which contain the underlay,

4) Make the CoQ₁₀-containing liposomes which contain an underlay by freeze drying or spray drying, or wiping off the moisture to obtain CoQ₁₀-containing preliposomes which contains spongiamine.

The CoQ₁₀-containing preliposomes of the present invention contain CoQ₁₀ at a concentration at of 0.2˜40% (W/W). After restoring by adding water, the concentration of the CoQ₁₀ is 0.1˜20% (W/W).

Suitable organic solvents that can be used according to the present invention include dichloromethane, trichloromethane, ether and ethanol.

The concentration of underlay used according to the present invention involved in the CoQ₁₀ preliposomes which contain spongiamine is 1˜80%.

Underlays that can be used according to the present invention are selected from one of the following materials: mannitol, glucose, sorbitol, sucrose, lactose, fucose, sodium chloride and polyvinylpyrrolidone.

The lipid components that can be used according the present invention include spongiamine and at least one of the following components: cholesterol, soy lecithin, yolk lecithin, hydrogenated lecithin, DSPC, DPPP, poloxamer, DMPC and non-ionic surfactant hire Brij.

The materials used in according to the present invention are all commercially available.

The CoQ₁₀-containing preliposomes which contain spongiamine according to the present invention not only have the same merit as the common liposomes in that they increase the stability of the drugs, facilitate the percutaneous absorption of drugs, and prolong the time of drug action In addition, the CoQ₁₀-containing preliposomes which contain spongiamine according to the present invention have the following merits:

1. The increased stability of CoQ₁₀-containing liposomes allow for longer storage times.

Because the the preliposomes are solid drugs, they overcome the shortcomings that the common liposomes have, such as congregating, sedimentating, fusing, leaking and so on.

2. The stability of the CoQ₁₀ is increased.

Because the the preliposomes are solid drugs, the present invention can be used to make unstable more stable in the solid stats than in the liquid state.

3. The percutaneous absorption of the CoQ₁₀ is increased.

The structure of the liposomes containing spongiamine according to the present invention facilitate the percutaneous absorption of drugs.

The CoQ₁₀-containing liposomes of the present invention can be mixed with other components at at random making them easier and more convenient to formulate into cosmetics.

Generally, for cosmetics which contain liposomes there is a certain range of the liposome volume. If the contains of liposomes exceed the range, characteristics of the cosmetics will be affected, such as viscosity, flow property, viscosity, the content of the active component and so on. Furthermore, certain cosmetics require different amounts of the CoQ₁₀. Before use, water can be added to the CoQ₁₀-containing preliposomes which contain spongiamine according to the present invention on demand, so as to provide liposomes which have different drug contents to meet different cosmetic prescriptions.

### EXAMPLES

### Example 1

In this example, 120 g of CoQ₁₀, 50 g of spongiamine. 50 g of yolk lecithin, 100 g of cholesterol, 100 g of sucrose, were combined with enough PBS (pH 7.4) to produce a volume of 1000 ml.

The CoQ₁₀, spongiamine, yolk lecithin and cholesterol from the above prescription were put into a triangle flask, heated to cause fusion, and stored in a water bath at 80° C. for further use. 800 ml of PBS (pH 7.4) was used to dissolve the 140 g of sucrose. The dissolved solution was filtered and heated in a water bath to reach the same temperature with the liposome solution. The sucrose solution was mixed with the liposome solution by surging and cooled. Enough PBS (pH 7.4) was added to produce 1000 ml of the mixed solution A high pressure homogeneous management (50 MPa of high pressure, 10 MPa of low pressure) was used to obtain a liposome suspension solution. After spray drying, a fluid CoQ₁₀-containing preliposomes which contained spongiamine was obtained.

### Example 2

In this example, 30 g of CoQ₁₀, 50 g of spongiamine, 30 g of soy lecithin, 100 g of cholesterol, 40 g of poloxamer F₅₈, 200 g of glucose, and 200 ml of chloral, were combined with enough PBS (pH 7.4) to produce a volume of 1000 ml.

The CoQ₁₀, spongiamine, soy lecithin, poloxamer F₆₈ and cholesterol from the above prescription were put into a 1000 ml rocked flask and the chloral was used to dissolve the lipid components. The resulting mixture was subject to membrane evaporation in a water bath at 25˜40° C. to make the lipid form a membrane layer at the bottom of the rocked flask. 800 ml of PBS (pH 7.4) was used to dissolve the 200 g of glucose. The solution was filtered and added to the flask containing the lipid membrane for hydration thereof using surging. Enough PBS (pH 7.4) was added to produce 1000 ml of mixed solution which was subject to ultrasonic treatment (output 4, duty cycle 50%, time 10 mins) to produce a liposome suspension solution. After freeze drying (temperature at −50° C. the degree of vacuum is 50 millitorr), a kind of loose CoQ₁₀-containing preliposomes which contain spongiamine was obtained.

### Example 3

In this example, 50 g of CoQ₁₀, 50 g of sponge 60 g of hydrogenated lecithin, 40 g of cholesterol, 50 g of poloxamer F₆₈, and 80 g of fucose, 200 ml of ether, were combined with enough PBS (pH 7.4) to produce a volume of 1000 ml.

The CoQ₁₀, spongiamine, hydrogenated lecithin, poloxamer F₆₈ and cholesterol from the above prescription were put into a 500 ml of triangle flask and the ether was added to dissolve the lipid components for further use. 800 ml of PBS (pH 7.4) was used to dissolve the 80 g of fucose. The fucose solution was filtered and put into the triangle flask which was stored in a water bath at 30˜60° C., and mixed round by magnetic force at the speed of 200˜1000 rpm. After the organic solvent was evaporation a liposome suspension solution was obtained and freeze dried (temperature at −50° C., the degree of vacuum is 50 millitorr) to produce a kind of loose CoQ₁₀-containing preliposomes which contain spongiamine.

### Example 4

### Test of Stability

Samples of the three batches of CoQ₁₀-containing preliposomes which contain spongiamine and a common CoQ₁₀-containing liposomes (the liposomes suspension before drying) were stored separately at a temperature of 40° C. and at a relative humidity level of 75%. After 0, 1, 2 and 3 months, High Performance Liquid Chromatography (BPLC) was used to test the content of CoQ₁₀ in the preliposomes and the common liposomes. The content of 0 month CoQ₁₀ in the preliposomes and the common liposomes was used as 100% to compare the content of drug at other times with the above mentioned content of CoQ₁₀, and calculate the percent content of drug as the time goes by.

Table 1 lists the stability comparing results of the content of CoQ₁₀ in the preliposomes and the common liposomes.

TABLE 1

The change percent of the
content of CoQ₁₀ (%)

| Time (mo) | 0 | 1 | 2 | 3 |
|---|---|---|---|---|
| Common | 100.00 | 93.32 | 88.03 | 83.50 |
| liposomes | | | | |
| Preliposomes | 100.00 | 99.86 | 99.53 | 98.76 |

The results show that the content of the drug contained in the common liposomes decreased along with the time while the content of the drug contained in the preliposomes did not decrease along with the time significantly. This indicates that the CoQ₁₀-containing preliposomes which contain spongiamine could evidently improve the stability of drugs.

1. CoQ₁₀-containing preliposomes, which contain spongiamine in the liposome structures.

2. CoQ₁₀-containing preliposomes according to claim 1, wherein the preliposomes are granular and a lyophilized solid.

3. CoQ₁₀-containing preliposomes of according to claim 1, wherein the preliposomes contain CoQ₁₀ at a concentration of 0.2˜40% (W/W).

4. CoQ₁₀-containing preliposomes according to claim 1, wherein the preliposomes contain spongiamine at a concentration of 0.1˜20% (W/W).

5. CoQ₁₀-containing preliposomes according to claim 1, further containing another lipid component.

6. A method of preparing the CoQ₁₀-containing preliposomes of claim 1 which comprises:
prepare a lipid solution by one of: a) melting CoQ₁₀ and spongiamine: and b) dissolving CoQ₁₀ and spongiamine in an organic solvent: and
applying the lipid solution to an underlay to produce the CoQ₁₀-containing preliposomes which contain spongiamine,

7. The method of claim 6, wherein the underlay comprises at least one of mannitol, glucose, sorbitol, sucrose, lactose, fucose, sodium chloride and polyvinylpyrrolidone.

8. A pharmaceutical composition that comprises the CoQ₁₀-containing preliposomes of claim 1.

9. A cosmetic composition that comprises the CoQ₁₀-containing preliposomes of claim 1.

10. CoQ₁₀-containing preliposomes according to claim 5, wherein the another lipid component comprises at least one of cholesterol, soy lecithin, yolk lecithin, hydrogenated lecithin, DSPC, DPPP, poloxamer, DMPC and a non-ionic surfactant.

11. The method of claim 6, wherein the lipid solution is applied to the underlay using a fluidized bed and the organic solvent is evaporated in the fluidized bed.

12. The method of claim 6, wherein the lipid solution is applied to the underlay by one of a membrane disperse process, a melt process and an infuse process.

13. The method of claim 6, wherein after the lipid solution is applied to the underlay the resulting mixture is subject to at least one of a freeze drying process, a spray drying process and a process of wiping moisture off the underlay.

14. The method of claim 13, wherein the underlay comprises at least one of mannitol, glucose, sorbitol, sucrose, lactose, fucose, sodium chloride and polyvinylpyrrolidone.

15. CoQ₁₀-containing preliposomes according to claim 2, wherein the preliposomes contain CoQ₁₀ at a concentration of 0.2˜40% (W/W).

16. CoQ₁₀-containing preliposomes according to claim 2, wherein the preliposomes contain spongiamine at a concentration of 0.1˜20% (W/W).

17. CoQ₁₀-containing preliposomes according to claim 2, further containing another lipid component.

18. CoQ₁₀-containing preliposomes according to claim 17, wherein the another lipid component comprises at least one of cholesterol, soy lecithin, yolk lecithin, hydrogenated lecithin, DSPC, DPPP, poloxamer, DMPC and a non-ionic surfactant.
